SEQUENCE LISTING (8 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 17 aa, synthetic construct
SFGCRFGTCTKISPQGY

SEQ ID NO: 3 - protein, 22 aa, synthetic construct
SFGCRFGTCTVAPRSKISPQGY

SEQ ID NO: 4 - protein, 27 aa, synthetic construct
SFGCRFGTCTKDKDNVAPRSKISPQGY

SEQ ID NO: 5 - protein, 32 aa, synthetic construct
SFGCRFGTCTYQFTDKDKDNVAPRSKISPQGY

SEQ ID NO: 6 - protein, 40 aa, synthetic construct
SFGCRFGTCTVQKLAHQIYQFTDKDKDNVAPRSKISPQGY

SEQ ID NO: 7 - protein, 40 aa, Homo sapiens; identical to SEQ ID NO: 6

SEQ ID NO: 8 - protein, 31 aa, Homo sapiens
TVQKLAHQIYQFTDKDKDNVAPRSKISPQGY

(19) United States
(12) Patent Application Publication
(10) Pub. No.: US 2026/0077017 A1
(43) Pub. Date: Mar. 19, 2026

(54) ADRENOMEDULLIN ANALOGS AND METHODS OF USE THEREOF

(71) Applicant: The Board of Trustees of the Leland Stanford Junior University, Stanford, CA (US)

(72) Inventors: Anca M. Pasca, Redwood City, CA (US); Dhriti Nagar, Redwood CIty, CA (US); Yusuke Hori, Stanford, CA (US); Alyssa Puno, Redwood CIty, CA (US)

(21) Appl. No.: 19/283,000

(22) Filed: Jul. 28, 2025

Related U.S. Application Data
(60) Provisional application No. 63/676,776, filed on Jul. 29, 2024.

(51) Int. Cl. A61K 38/22; A61P 25/28

(52) U.S. Cl. CPC A61K 38/22; A61P 25/28

(57) ABSTRACT

Adrenomedullin (ADM) is shown to be protective against damage to neurons as a result of hypoxic brain injuries, radiation brain injuries, and to prevent neurodevelopmental injury in encephalopathy of prematurity; 22q11.2 deletion syndrome, e.g. DiGeorge Syndrome; and other neurologic diseases associated with mitochondrial dysfunction. Modified ADM peptides are provided, which are stabilized relative to the native peptide. In an embodiment, a therapeutic composition is provided, comprising an ADM analog of the disclosure, and a pharmaceutically acceptable excipient. In an embodiment, a method is provided for treating or preventing neurologic damage in an individual, the method comprising administering an effective dose of ADM.

### GOVERNMENT SUPPORT RESEARCH

This invention was made with Government support under contract MH128352 awarded by the National Institutes of Health. The Government has certain rights in the invention.

### REFERENCE TO ELECTRONIC SEQUENCE LISTING

The contents of the electronic sequence listing (STAN-2205 Seqlist.xml; Size: 8,140 bytes; and Date of Creation: Jun. 23, 2025) is herein incorporated by reference in its entirety.

### BACKGROUND

Adrenomedullin (ADM or AM) is a circulating peptide hormone with pleiotropic effects. It was initially identified as a vasodilator. Other effects of AM include stimulating the growth of new blood vessels (angiogenesis) and increasing the tolerance of cells to oxidative stress and hypoxic injury. Adrenomedullin is seen as a positive influence in diseases such as hypertension, myocardial infarction, chronic obstructive pulmonary disease and other cardiovascular diseases.

Adrenomedullin consists of 52 amino acids, has 1 intramolecular disulfide bond, and shows a slight homology with the calcitonin gene-related peptide (CGRP). The precursor, called preproadrenomedullin, consists of 185 amino acids and can be cleaved by plasma kallikrein at the Lys-Arg and Arg-Arg sites. Mature human ADM is activated to form a 52 amino acid, 6-amino acid ring, which has a short plasma half-life of about 22 min.

ADM exerts its actions through combinations of the calcitonin receptor like receptor (CALCRL) or CLR; and either receptor activity-modifying protein 2 (RAMP2) or RAMP3. Both transduce the hormone binding to intracellular signaling via second messenger cascades. The interaction of both CALCRL and RAMP at the membrane is required for ADM activity.

### SUMMARY

Adrenomedullin (ADM) and ADM analogs are shown to be protective against damage to neurons as a result of hypoxic brain injuries, radiation brain injuries, and to prevent neurodevelopmental injury in encephalopathy of prematurity; 22q11.2 deletion syndrome, e.g. DiGeorge Syndrome; and other neurologic diseases associated with mitochondrial dysfunction.

In an embodiment of the invention, ADM analog peptides are provided. These analogs are stabilized relative to the native peptide, which is provided herein for reference as SEQ ID NO:1. The analogs comprise at least residues 13-22 and 45-52 of SEQ ID NO:1. In some embodiments the analog sequence is modified relative to SEQ ID NO:1, provided that the presence of residues 147, P49, G51 and Y52 is maintained. In native ADM, the cysteines at residues C16 and C21 are disulfide bonded. In the analogs, including SEQ ID NO:1, the corresponding disulfide bridge is substituted by a xylene bridge, for example using o-dibromoxylene. The xylyl modified analogs have increased stability relative to native ADM. Exemplary analogs are provided in SEQ ID NO:2, 3, 4, 5, and 6. The analogs may be amidated at the C-terminus. The analogs may be acylated at the N-terminus.

In an embodiment, a therapeutic composition is provided, comprising an ADM analog of the disclosure, and a pharmaceutically acceptable excipient. The composition may be provided in a unit dose, comprising an effective dose of the ADM peptide. The dose may be effective in treating or protecting neurons against hypoxic brain injuries.

In an embodiment, a method is provided for treating or preventing neurologic damage in an individual, the method comprising administering an effective dose of ADM. In some embodiments the ADM is an ADM analog as described herein. In some embodiments the ADM is a native human ADM peptide. The ADM may be administered prior to a treatment expected to cause neurologic damage, e.g. radiation therapy. The ADM may be administered following a determination of a condition in the individual associated with neurologic damage, e.g. hypoxia, a condition associated with mitochondrial dysfunction, and the like. In some embodiments the neurologic damage is associated with radiation therapy. In some embodiments the neurologic damage is associated with hypoxia. In some embodiment the neurologic damage is associated with mitochondrial dysfunction, including without limitation 22q11.2 deletion syndrome. In other embodiments the analogs are administered in an in vitro setting, e.g. in a culture comprising primary neurons or organoids, e.g. for research or screening purposes.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1B. FIG. 1A) Analysis of neural cell cytotoxicity in brain organoids exposed to hypoxia. Compound 1 is wild-type adrenomedullin. It is seen that ADM is protective against cytotoxic damage. FIG. 1B) Analysis of neuronal cell redox state in brain organoids exposed to hypoxia. It is seen that ADM normalizes the redox state of the cells.

FIG. 2. Toxilight assay for IR2 (irradiation experiment 2). Time course of effects of ADM on radiation injury in organoids (30 Gy) after 48 hr. The toxilight assay measures toxicity in mammalian cells in culture based on the bioluminescent measurement of adenylate kinase. A loss of cell integrity increases release of AK. There is a significant increase in injury in 30Gy-exposed versus control and partial rescue by ADM (0.5 UM).

FIG. 3. Sequences of ADM analogs. Wild-type ADM is provided as SEQ ID NO:1. Analog P12 is SEQ ID NO:2. Analog P13 is SEQ ID NO:3. Analog P14 is SEQ ID NO:4. Analog P15 is SEQ ID NO: 5. Analog P16 is SEQ ID NO: 6.

FIGS. 4A-4E. Structure of ADM analogs P12 (SEQ ID NO:2), P13 (SEQ ID NO:3), P14 (SEQ ID NO:4), P15 (SEQ ID NO:5), and P16 (SEQ ID NO:6).

FIG. 5. Effects of ADM and ADM analogs on cell death under hypoxic conditions. cPARP expression decreases in ADM with P12-P16. P5 is a known agonist, as control.

FIGS. 6A-6G. Defects in mitochondrial morphology in 22q11.2DS human fetal brain neurons are rescued by ADM. A, B, C: Upper row: mitochondrial tagging in brain cells from human fetal tissue at 15 weeks 6 days, in control, 22q11.2 deletion and 22q11.2 deletion +ADM conditions; D, E, F, G: lower row shows quantified changes in mitochondrial area, perimeter, form factor and aspect ratio in the 3 conditions mentioned above.

FIGS. 7A-7E. Upper row: mitochondrial tagging in brain cells from organoids at day 100 in culture, in control, 22q11.2 deletion, and 22q11.2 deletion +ADM conditions; lower row shows quantified changes in mitochondrial perimeter and form factor in the 3 conditions mentioned above.

FIGS. 8A-8C. Defects in mitochondria morphology in 22q11.2DS human cortical brain organoids are rescued by ADM analogs. FIG. 8A) Representative micrographs showing mitochondrial morphology visualized using pkmitodeepred dye in monolayer cultures obtained from human cortical brain organoids (hCO) at day 100. Quantification of FIG. 8B) Mean mitochondrial aspect ratio and FIG. 8C) mean mitochondrial form factor.

FIGS. 9A-9C. Defects in mitochondria morphology in 22q11.2DS human fetal brain primary neurons are rescued by ADM analogs. FIG. 9A) Representative micrographs showing mitochondrial morphology visualized using pkmitodeepred dye in primary cultures obtained from human fetal brain tissue (hFT) at 19w4d. Quantification of FIG. 9B) Mean mitochondrial aspect ratio and FIG. 9C) mean mitochondrial form factor.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Comparable cell” shall mean a cell whose type is identical to that of another cell to which it is compared. Examples of comparable cells are cells from the same cell line.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent. In some embodiments treatment prevents neurologic damage associated with a particular treatment, e.g. hypoxia, radiation, etc.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with hypoxia, mitochondrial dysfunction, radiation, etc. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to prevent, treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

“Inhibiting” the expression of a gene in a cell shall mean either lessening the degree to which the gene is expressed, or preventing such expression entirely. “Specifically inhibit” the expression of a protein shall mean to inhibit that protein's expression (a) more than the expression of any other protein, or (b) more than the expression of all but 10 or fewer other proteins.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Anti-sense nucleic acid” shall mean any nucleic acid which, when introduced into a cell, specifically hybridizes to at least a portion of an mRNA in the cell encoding a protein (“target protein”) whose expression is to be inhibited, and thereby inhibits the target protein's expression.

“Subject” or “patient” shall mean any animal, such as a human, non-human primate, mouse, rat, guinea pig or rabbit.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having a disease of interest. Subjects may be human, but also include other mammals, particularly those mammals useful as laboratory models for human disease, e.g. mouse, rat, etc.

The definition of an appropriate patient sample encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, etc. A sample if interest in bronchial lavage sample. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient. A biological sample comprising a sample cell from a patient can also include normal, non-diseased cells.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect. First therapeutic agents contemplated for use with the methods of the present invention include any other agent for use in the treatment of the specific neurologic conditions of interest.

“Concomitant administration” of a known therapeutic agent with a pharmaceutical composition of the present invention means administration of the therapeutic agent and ADM agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” or “unit dose” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” or “formulation” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning. The methods of the invention also include the use of factor combinations that consist, or consist essentially of the desired factors.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.” A unit dose may comprise an effective amount of an active agent for a single administration.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

### Conditions for Treatment

Mitochondrial dysfunction. Neurons are metabolically active cells with high energy demands at locations distant from the cell body. As a result, these cells are particularly dependent on mitochondrial function, as reflected by the observation that diseases of mitochondrial dysfunction often have a neurodegenerative component. Recent discoveries have highlighted that neurons are reliant particularly on the dynamic properties of mitochondria. Mitochondria are dynamic organelles by several criteria. They engage in repeated cycles of fusion and fission, which serve to intermix the lipids and contents of a population of mitochondria. In addition, mitochondria are actively recruited to subcellular sites, such as the axonal and dendritic processes of neurons. Finally, the quality of a mitochondrial population is maintained through mitophagy, a form of autophagy in which defective mitochondria are selectively degraded. Defects in the key features of mitochondrial dynamics, such as mitochondrial fusion, fission, transport and mitophagy are associated with neurodegenerative disorder. Several major neurodegenerative disorders—including Parkinson's, Alzheimer's and Huntington's disease—involve disruption of mitochondrial dynamics.

22q11 deletion syndromes. 22q11 deletion syndromes include Shprintzen-Goldberg syndrome, velocardiofacial syndrome, Cayler cardiofacial syndrome, Sedlackova syndrome, conotruncal anomaly face syndrome, and DiGeorge Syndrome (DGS).

Microdeletion of a 3 Mb region encompassing 45 protein-coding genes at chromosome 22q11.2 (22q11.2DS) predisposes individuals to multiple neurodevelopmental disorders and is one of the greatest genetic risk factors for schizophrenia. Defective mitochondrial function has been hypothesized to contribute to 22q11.2DS pathogenesis. The 22q13.1-33 region contains 6 genes associated with mitochondrial function, which could cause many of the clinical findings in through disruption of mitochondrial function.

About 90% of DGS cases are a result of a deletion in chromosome 22, more specifically on the long arm (q) at the 11.2 locus (22q11.2). Most of these mutations arise de novo with no genetic abnormalities noted in the genome of the parents. Researchers have identified over 90 different genes at this locus. The most studied of these genes is T-box transcription factor 1 (TBX1), which correlates with severe defects in the development of the heart, thymus, and parathyroid glands of mouse models. TBX1 also correlates with neuromicrovascular anomalies, which may be responsible for the behavioral and developmental abnormalities seen in DGS.

Features of 22q11 deletion syndromes include an absent or hypoplastic thymus, cardiac abnormalities, hypocalcemia, and parathyroid hypoplasia. Some patients may have a mild to moderate immune deficiency, and the majority of patients have cardiac anomalies. Other features include palatal, renal, ocular, and gastrointestinal anomalies. Skeletal defects, psychiatric disease, and developmental delay are also of concern. Delay in motor development is a common presenting feature first recognized by parents who notice delays in rolling over, sitting up, or other infant milestones. These findings can be associated with delayed speech development and learning disabilities. Later in life, abnormal behavior in the setting of poor developmental history may be the chief presenting symptom.

A clinician makes a definitive diagnosis in individuals with a microdeletion of chromosome 22 at the 22q11.2 locus. Microdeletions are detected by fluorescence in situ hybridization (FISH), multiplex ligation-dependent probe amplification (MLPA), single nucleotide polymorphism (SNP) array, comparative genomic hybridization (CGH) microarray, or quantitative polymerase chain reaction (qPCR).

Encephalopathy of prematurity. Encephalopathy of prematurity represents the leading risk factor for epilepsy and childhood-onset neuropsychiatric diseases, including autism spectrum disorders (ASD) and attention deficit hyperactivity disorder (ADHD). The prevalence of neuropsychiatric diseases is highest in individuals born extremely preterm at <28 postconceptional weeks (PCW) and correlates with the degree of prematurity. While the pathophysiology of encephalopathy of prematurity is complex, clinical studies identify perinatal hypoxic events as one of the most important environmental risk factors in the perinatal period, along with inflammation.

Encephalopathy of prematurity (EOP) is a histologically defined condition common in preterm infants characterized by cerebral white matter injury/periventricular leukomalacia with a variety of associated neuronal/axonal deficits. The fetal/maternal and neonatal conditions leading to EOP are those typically associated with cerebral ischemia and systemic infection/inflammation. The diagnosis of EOP relies heavily on EEG and neuroimaging studies, mainly head ultrasound and EEG. Infants with EOP have a high incidence of chronic neurodevelopmental impairment in a variety of areas: motor coordination, visual function, cognitive function, language ability, socialization, and behavior. Clinical management of preterm infants during the neonatal period may influence outcome, and relevant elements include administration of steroids prior to delivery as well as management of oxygen levels, carbon dioxide levels, blood pressure, glucose levels, seizures, and patent ductus arteriosus. Neuroprotective strategies that might mitigate the effects of EOP are currently under evaluation.

Developmental injury of cortical interneurons is increasingly recognized as a major contributor to the pathophysiology of neuropsychiatric diseases associated with encephalopathy of prematurity. This is notable because: (i) the second half of in utero human prenatal brain development corresponds to the peak timing of migration of cortical interneurons from the medial ganglionic eminences (MGE) into the dorsal forebrain, (ii) histological studies show a significant decrease in the number of cortical interneurons within the cerebral cortex of individuals born extremely premature, and (iii) perinatal episodes of hypoxia and/or inflammation are most common in extremely preterm infants.

Hypoxic Brain Injury. The brain consumes a significant amount of energy compared to its weight and size. The term “anoxia” refers to the complete lack of oxygen delivery to an organ. The term “hypoxia” applies when an organ experiences oxygen delivery that is insufficient to meet the metabolic needs of the tissue. The pathological mechanisms precipitated by cerebral hypoxia or anoxia are similar, and the terms “anoxic brain injury” and “hypoxic brain injury” are sometimes used interchangeably. The brain consumes a significant amount of energy compared to its weight and size. It is highly metabolically active and exquisitely sensitive to hypoxia and hypoperfusion. Cellular injury can begin within minutes, and permanent brain injury will follow if prompt intervention does not occur.

The brain depends on a constant energy supply provided by glucose and oxygen but is unable to store energy. With the cessation of blood flow, intracellular production of adenosine triphosphate is diminished. This results in the dysfunction of energy-dependent ion channels, which contributes to intracellular sodium accumulation and cytotoxic edema. Ongoing ischemia results in the release of glutamate, an excitatory neurotransmitter, which promotes calcium influx through N-methyl-D-aspartate (NMDA) receptors. Calcium influx exacerbates neuronal injury by activating lytic enzymes, precipitating free radical formation, and interfering with mitochondrial function. This process, known as excitotoxicity, can ultimately lead to cell death.

The mechanisms that lead to delayed cell death following hypoxic-ischemic injury in the brain are complex. Ischemic cell death occurs via two different pathways: necrosis and apoptosis. During hypoxia-ischemia of the brain, acute energy failure leads to loss of ion homeostasis where intracellular sodium and calcium accumulate, creating osmotic swelling, which can lead to cell lysis. This process releases glutamate and free radicals, which are cytotoxic and exacerbate the injury. A secondary phase of neuronal death can occur hours later.

In the setting of brain anoxia due to cardiac arrest, resuscitation efforts, optimizing blood pressure, and maintaining systemic perfusion are critical components of treatment. Clinical management is focused on supportive care, treatment of the underlying cause of the hypoxia, and prevention of ongoing brain injury. A variety of neuroprotective strategies have been evaluated in an effort to prevent cell death by interrupting or attenuating the cascade of events by which hypoxia precipitates neuronal apoptosis and necrosis.

Radiotherapy is widely used to treat recurrent cancer, such as brain and breast carcinomas. Whole-brain radiotherapy (WBRT) is the treatment of choice for local recurrence after surgical resection of brain metastases. Yet, radiation to the brain may cause certain side effects. RIBI (Radiation-Induced Brain Injury) is a common complication caused by radiation treatment for head and neck tumors. The longer the radiation exposure, the more malignant the disease. Acute injury and subsequent long-term damage often lead to multifocal hypometabolism and persistent neuroinflammation of the brain

Acute RIBI often occurs days or weeks after irradiation, mainly due to cerebral edema, increased intracranial pressure, and transient neurological impairment caused by increased blood-brain barrier (BBB) permeability. Its main clinical manifestations include headache, nausea, vomiting, increased body temperature, disturbance of consciousness, and convulsions, which are generally recoverable. Early delayed RIBI is usually a temporary and reversible white matter injury that occurs after a few weeks to 3 months after brain radiotherapy and is mainly characterized by demyelinating lesions of oligodendrocytes with axonal edema. Its clinical manifestations include lethargy, nausea, and irritability, which can usually be cured after active treatment during this period.

Late delayed RIBI complicated with abnormal vascular changes and demyelination, and white matter necrosis often occurs 6 months after irradiation; this stage is commonly irreversible and progressive. Late RIBI (3 months to several years) is accompanied by local nerve tissue abnormalities and increased intracranial pressure, and its diagnosis, which is based on the clinical manifestations alone, is difficult to establish. During this stage, low-density areas of white matter increase with irregular enhancement effects on the computed tomography (CT) images, accompanied by diffusing edema around the lesion and varying degrees of space-occupying effects.

### ADM Peptides and Formulations

In an embodiment of the invention, modified ADM peptides are provided. These analogs are stabilized relative to the native peptide, provided herein for reference as SEQ ID NO:1. The analogs comprise at least residues 13-22 and 45-52 of SEQ ID NO:1. In some embodiments the sequence is modified, provided that the presence of residues 147, P49, G51 and Y52 is maintained (numbering relative to SEQ ID NO:1). In native ADM, the cysteines at residues C16 and C21 are disulfide bonded. In the analogs, the corresponding disulfide bridge is substituted by a xylene bridge, for example using o-dibromoxylene. The o-xylyl modified analogs have increased stability relative to native ADM, e.g. providing a longer half-life after administration, in plasma, or in a formulation. Exemplary analogs are provided in SEQ ID NO:2, 3, 4, 5, and 6. The analogs may be amidated at the C-terminus. The analogs may be acylated at the N-terminus.

Xylene, or dimethylbenzene, is an aromatic hydrocarbon with a benzene ring and two methyl substituents. A xylene bridge in a peptide is a structural linker containing this xylene moiety, replacing the disulfide (—S—S—) bond that typically forms between two cysteine residues. Xylene bridges can be introduced during the chemical synthesis of peptides, for example using dibromoxylene derivatives to react with appropriately functionalized lysine or cysteine residues to form the covalent xylene link. Techniques like CLIPS (Chemical Linkage of Peptides onto Scaffolds) can be employed for this purpose. See, for example, Beard et al. (2018) Bioorganic & Medicinal Chemistry vol. 26(11):3039.

Specific polypeptides of interest include, for example, those listed in Table 1:

TABLE 1

| | | Modifications |
|---|---|---|
| | | for Redox |
| Name | Sequence (Linear) | stabilization |
| ADM 1-52 | YRQSMNNFQGLRSFGCR | No |
| (SEQ ID NO: 1) | FGTCTVQKLAHQIYQFT | modification |
| | DKDKDNVAPRSKISPQG | |
| | Y | |
| ADM 13-52 | SFGCRFGTCTVQKLAHQ | No |
| (SEQ ID NO: 7) | IYQFTDKDKDNVAPRSK | modification |
| | ISPQGY | |
| ADM 22-52 | TVQKLAHQIYQFTDKDK | No |
| (SEQ ID NO: 8) | DNVAPRSKISPQGY | modification |
| ADM 13-22 | SFGCRFGTCTKISPQGY | BromoXylene |
| (cyclical) + | | (thioether) |
| ADM 45-52 | | |
| (linear) | | |
| (SEQ ID NO: 2) | | |
| ADM 13-22 | SFGCRFGTCTVAPRSKI | BromoXylene |
| (cyclical) + | SPQGY | (thioether) |
| ADM 40-52 | | |
| (linear) | | |
| (SEQ ID NO: 3) | | |
| ADM 13-22 | SFGCRFGTCTKDKDNVA | BromoXylene |
| (cyclical) + | PRSKISPQGY | (thioether) |
| ADM 35-52 | | |
| (linear) | | |
| (SEQ ID NO: 4) | | |
| ADM 13-22 | SFGCRFGTCTYQFTDKD | BromoXylene |
| (cyclical) + | KDNVAPRSKISPQGY | (thioether) |
| ADM 30-52 | | |
| (linear) | | |
| (SEQ ID NO: 5) | | |
| ADM 13-22 | SFGCRFGTCTVQKLAHQ | BromoXylene |
| (cyclical) + | IYQFTDKDKDNVAPRSK | (thioether) |
| ADM 23-52 | ISPQGY | |
| (linear) | | |
| (SEQ ID NO: 6) | | |

Relative to wild-type ADM, a peptide according to any of SEQ ID NO:2, 3, 4, 5, 6 can have increased stability, e.g. stability in a formulation, t_{1/2} following in vivo administration, and the like. For example the half-life may be increased by 50%, 75%, 2-fold, 3-fold, 4-fold, 5-fold, or more.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man, e.g. as set forth in SEQ ID NO:1.

By “protein variant” or “variant protein” or “variant polypeptide” herein is meant a protein that differs from a provided sequence by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. A variant polypeptide comprises at least one amino acid modification compared to the parent polypeptide, e.g. from about one to about ten amino acid modifications, and may be from about one to about five amino acid modifications compared to the parent. A variant may be at least about 99% identical to the reference protein, at least about 98% identical, at least about 97% identical, at least about 95% identical, at least about 90% identical.

The term “identity,” as used herein in reference to polypeptide or DNA sequences, refers to the sequence identity between two molecules. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “polypeptide,” “protein” or “peptide” refer to any chain of amino acid residues, regardless of its length or post-translational modification (e.g., glycosylation or phosphorylation).

By “parent polypeptide”, “parent protein”, “precursor polypeptide”, or “precursor protein” as used herein is meant an unmodified polypeptide that is subsequently modified to generate a variant polypeptide. A parent polypeptide may be a wild-type (or native) polypeptide. Parent polypeptide may refer to the polypeptide itself, compositions that comprise the parent polypeptide, or the amino acid sequence that encodes it.

Polypeptides may also include conservative modifications and substitutions at other positions of the cytokine (e.g. positions other than those involved in the cysteine engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gin, asn, ser, thr; Group II: cys, ser, tyr, thr; Group III: val, ile, leu, met, ala, phe; Group IV: lys, arg, his; Group V: phe, tyr, trp, his; and Group VI: asp, glu. In each instance, the introduction of additional modifications may be evaluated to minimize any increase in antigenicity of the modified polypeptide in the organism to which the modified polypeptide is to be administered.

In some embodiments, a polypeptide is conjugated to additional molecules to provide desired pharmacological properties such as extended half-life. In one embodiment, a polypeptide can be fused to the Fc domain of IgG, albumin, or other molecules to extend its half-life, e.g. by pegylation, glycosylation, and the like as known in the art. In some embodiments the polypeptide is conjugated to a polyethylene glycol molecules or “PEGylated.” The molecular weight of the PEG conjugated to the polypeptide ligand include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the polypeptide, or attached via a linker molecule. The processes and chemical reactions necessary to achieve PEGylation of biological compounds is well known in the art.

The polypeptide can be acetylated at the N-terminus, using methods known in the art, e.g. by enzymatic reaction with N-terminal acetyltransferase and, for example, acetyl COA. The polypeptide can be acetylated at one or more lysine residues, e.g. by enzymatic reaction with a lysine acetyltransferase. See, for example Choudhary et al. (2009). Science. 325(5942):834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The ortholog IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, a polypeptide can comprise a sequence that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

As described above, the proteins of the invention may exist as a part of a chimeric polypeptide. In addition to, or in place of, the heterologous polypeptides described above, a nucleic acid molecule of the invention can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo1, G418r), dihydrofolate reductase (DHFR), hygromycin-B-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

### Administration

In some embodiments, an effective dose of an ADM analog polypeptide as disclosed herein, e.g. a peptide according to any of SEQ ID NO:2, 3, 4, 5,6 is administered to an individual to treat damage to neurons as a result of hypoxic brain injuries, radiation brain injuries, and to prevent neurodevelopmental injury in encephalopathy of prematurity; 22q11.2 deletion syndrome, e.g. DiGeorge Syndrome; and other neurologic diseases associated with mitochondrial dysfunction. In some embodiments, an effective dose of an ADM polypeptide is administered to prevent damage to neurons as a result of hypoxic brain injuries, radiation brain injuries, and to prevent neurodevelopmental injury in encephalopathy of prematurity; 22q11.2 deletion syndrome, e.g. DiGeorge Syndrome; and other neurologic diseases associated with mitochondrial dysfunction.

Individuals selected for treatment may be neonatal, including, for example, premature neonates. Individuals selected for treatment may be infants, e.g. from birth to about 1 year of age, may be children, e.g. from about 1 to 18 years of age, and may be adults, including aged adults, depending on the specific condition that is treated.

An effective dose of an ADM polypeptide may range up to about 30 mg/kg, up to about 20 mg/kg, up to about 10 mg/kg, up to about 5 mg/kg; up to about 1 mg/kg, up to about 0.5 mg/kg; up to about 0.1 mg/kg; up to about 0.05 mg/kg; and may be at least about 1 μg/kg, at least about 5 g/kg, at least about 10 μg/kg, at least about 50 μg/kg, where the dose may vary with the specific analog and recipient.

The ADM agents may be administered one or a plurality of days, and in some embodiments is administered daily, every two days, semi-weekly, weekly, etc. for a period of from about 1, about 2, about 3, about 4, about 5, about 6, about 7 or more weeks, up to a chronic maintenance level of dosing. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved.

According to the present invention, compositions can be administered by parenteral, topical, intravenous, oral, subcutaneous, intraarterial, intracranial, intraperitoneal, intranasal, aerosol, or intramuscular means. The most typical route of administration is intravenous although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. ADM polypeptides can be administered in the form of a depot injection or implant preparation which can be formulated in such a manner as to permit a sustained release of the active ingredient. An exemplary composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and Jun. 2, 2005 antioxidants (e.g., parabens, vitamins E and C, and ascorbic acid), anti-caking agents, coating agents, and chelating agents (e.g., EDTA).

In some embodiments, a dose of the agent is provided in an implant, e.g. a matrix or scaffold, osmotic pump or other delivery device, etc., for localized delivery of the factor. The effective dose may be determined based on the specific tissue, rate of release from the implant, size of the implant, and the like. and may be empirically determined by one of skill in the art. The dose may provide for biological activity equivalent to 1 μg, 10 μg, 100 μg, 1 mg, 5 mg, 10 mg, 25 mg, 50 mg, 75 mg, 100 mg, 250 mg, 500 mg, 750 mg, 1 g of ADM. The dose may be administered at a single time point, e.g. as a single implant; or may be fractionated, e.g. delivered in a microneedle configuration. The dose may be administered, once, two, three time, 4 times, 5 times, 10 times, or more as required to achieve the desired effect, and administration may be daily, every 2 days, every 3 days, every 4 days, weekly, bi-weekly, monthly, or more.

In some approaches, the agents are administered directly to an injured site to treat a neurological condition, see, e.g., Morizane et al., (2008), Cell Tissue Res., 331(1):323-326; Coutts and Keirstead (2008), Exp. Neurol., 209(2):368-377; Goswami and Rao (2007), Drugs, 10(10):713-719.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

The pharmaceutical compositions are generally formulated as sterile, substantially isotonic and in full compliance with all Good Manufacturing Practice (GMP) regulations of the U.S. Food and Drug Administration. Preferably, a therapeutically effective dose will provide therapeutic benefit without causing substantial toxicity.

Toxicity of the polypeptides described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. A kit may comprise, for example, one or more unit doses of an ADM analog as disclosed herein. The kit can further contain a least one additional reagent. Kits typically include a label indicating the intended use of the contents of the kit. The term label includes any writing, or recorded material supplied on or with the kit, or which otherwise accompanies the kit.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Results

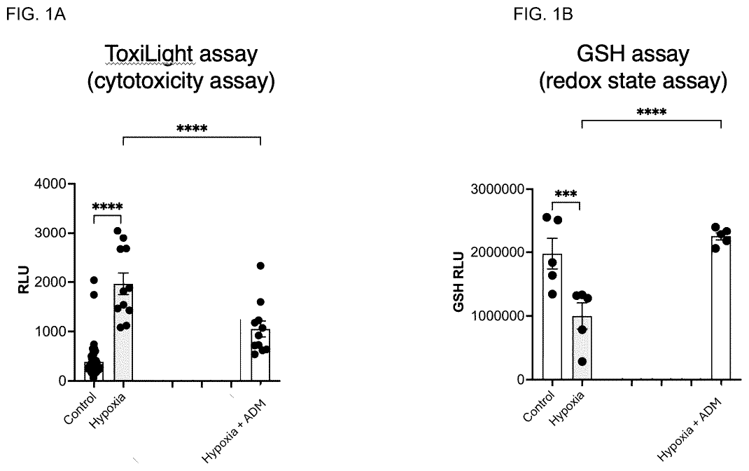

Protective effect of ADM in hypoxia. Brain organoids were subjected to hypoxic conditions, as shown in FIGS. 1A and 1B, in the absence or presence of adrenomeddulin (ADM) where adrenomedullin is labeled as compound #1. A. Effects of ADM on radiation injury in organoids (30 Gy) after 48 hr is shown, following 1 exposure. There was a significant increase of injury in 30 Gy-exposed versus control organoids, and partial rescue by ADM. B. Effects of ADM and ADM analogs on cell death under hypoxic conditions.

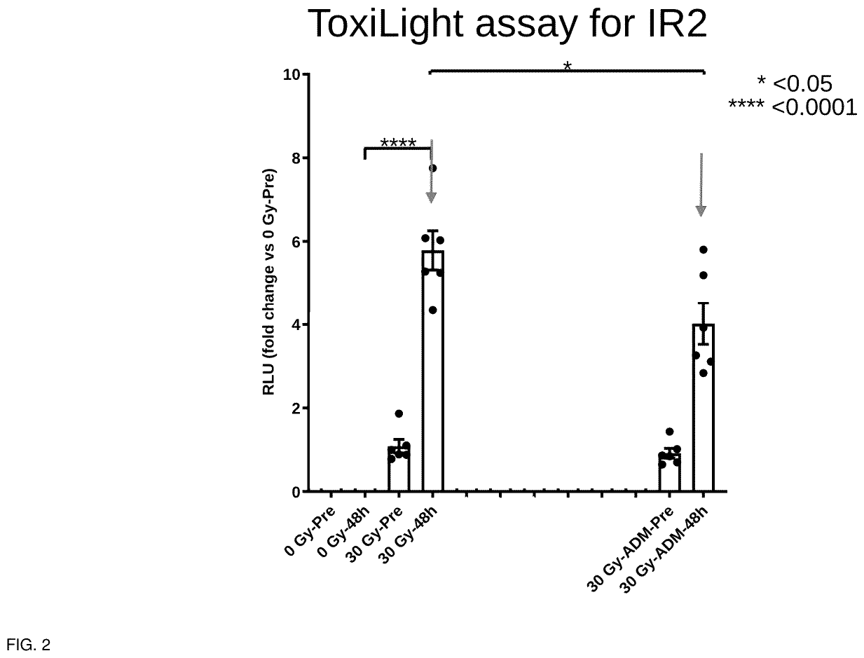

FIG. 2 shows cytotoxicity following radiation damage, and partial protection by wild-type ADM.

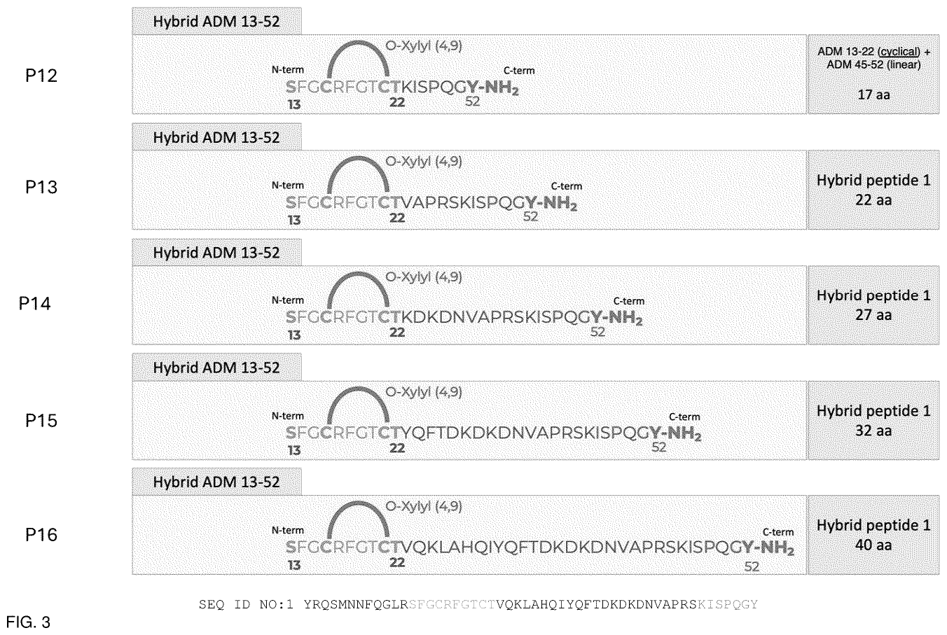

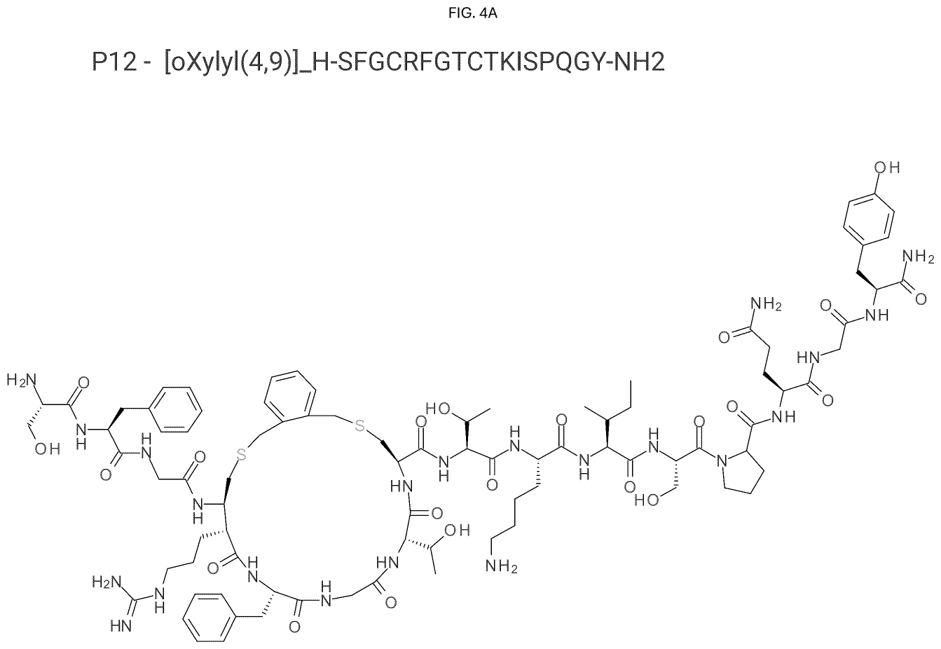

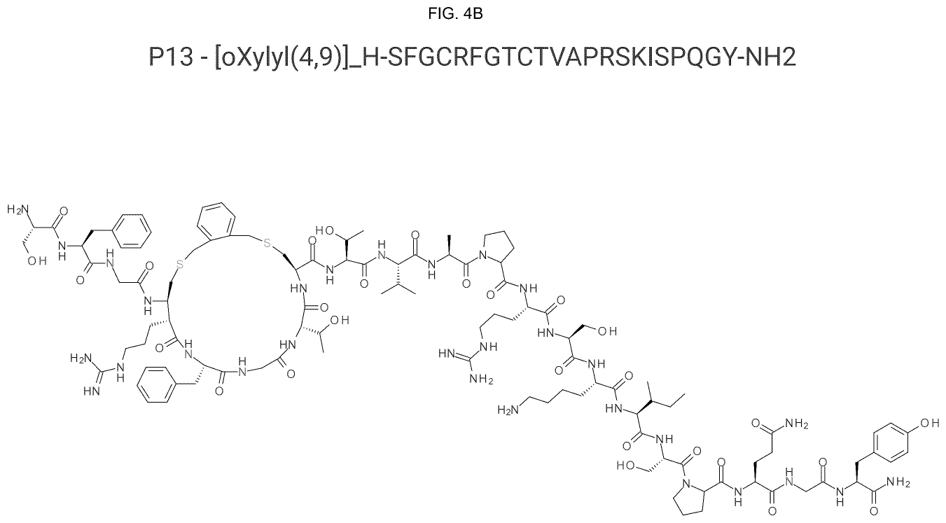

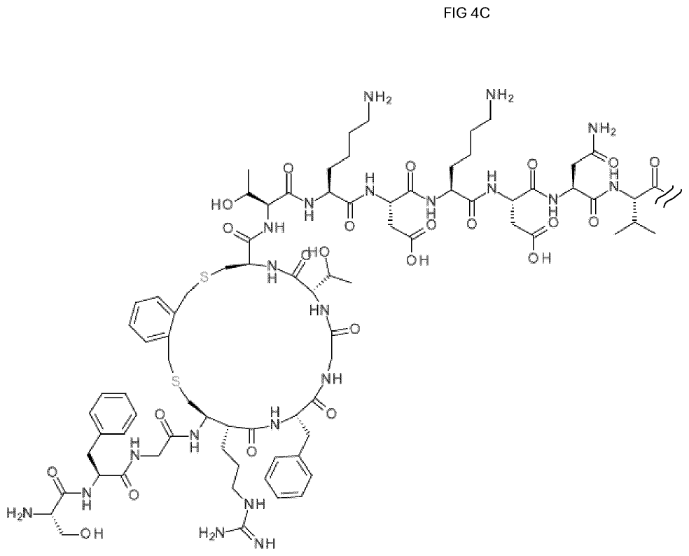

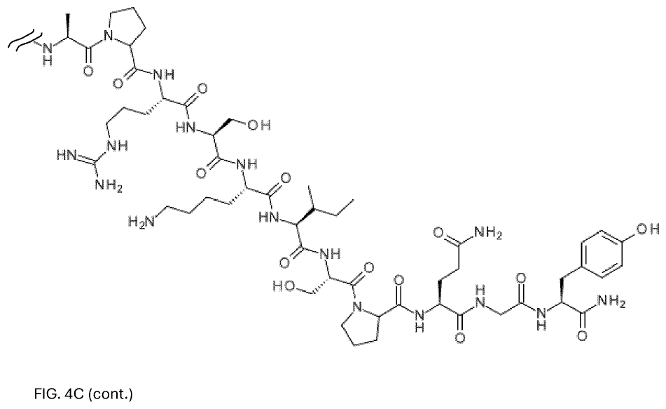

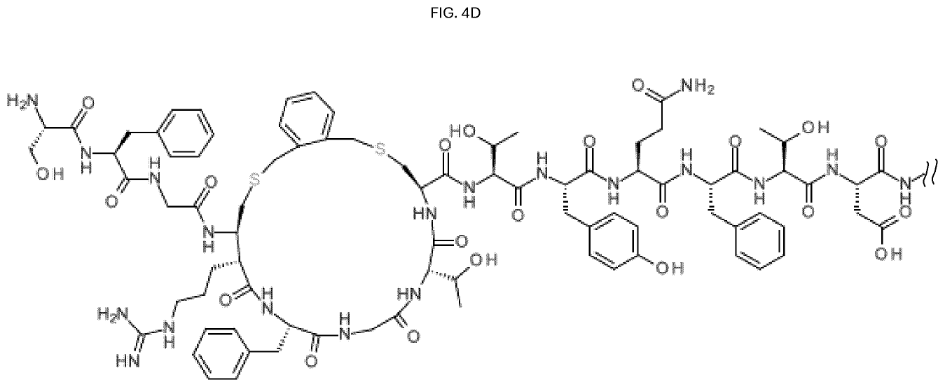

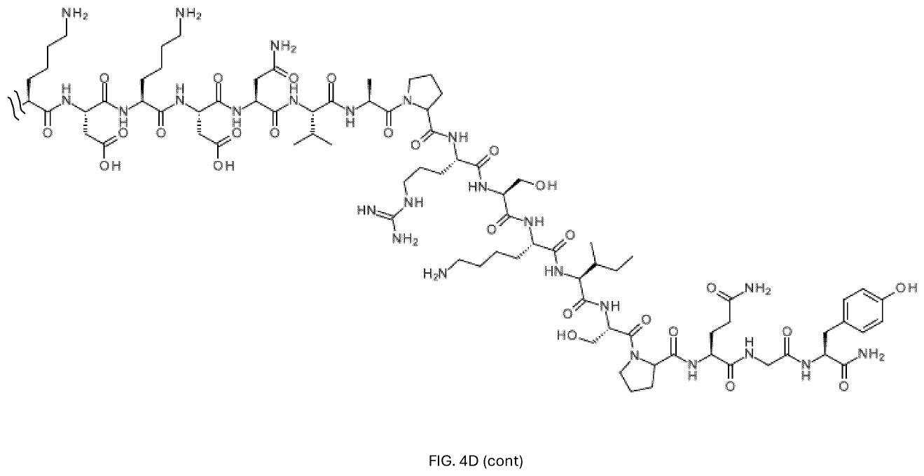

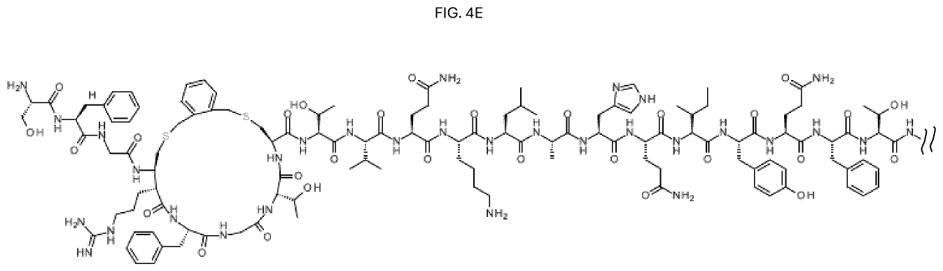

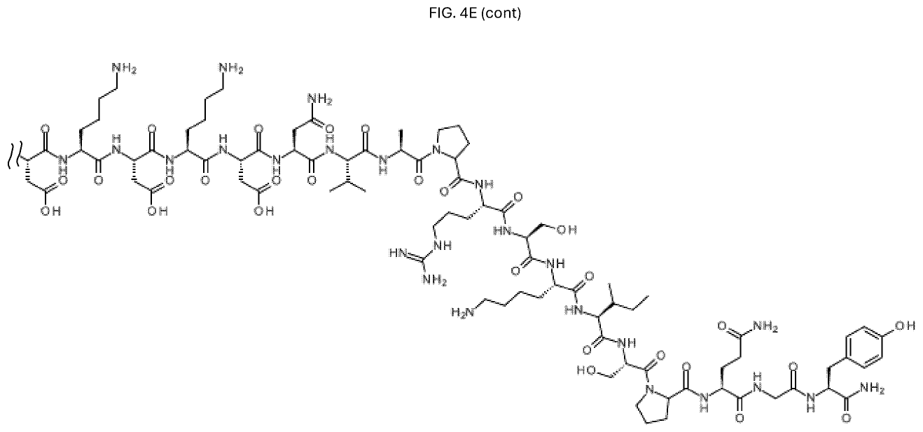

The sequence and structures of ADM analogs are shown in FIG. 3 and FIGS. 4A-4E.

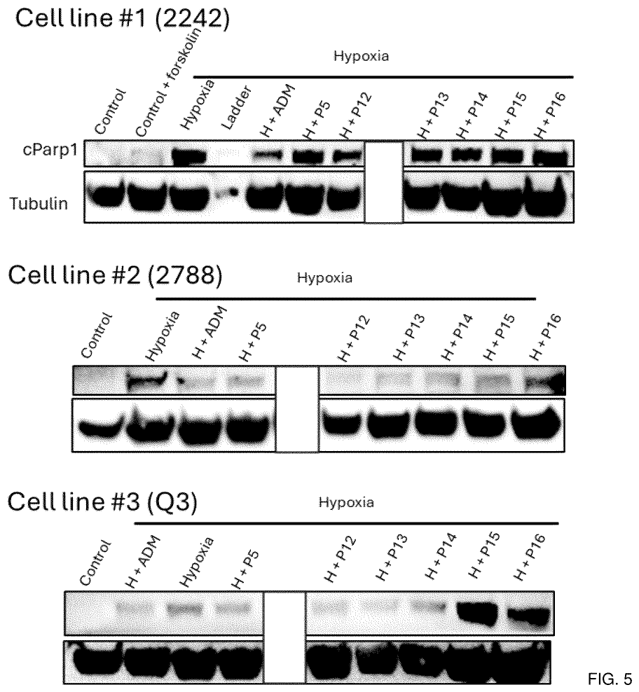

Shown in FIG. 5, under hypoxic conditions there is an increase in cPARP expression. In the presence of ADM or the ADM analogs, P12, P13, P14, P15 and P16 there is a decrease in cPARP overexpression. P5 is a known agonist, included as a control. Table 1 below presents quantification of data from FIG. 5.

| | | | Averaged | Relative |
|---|---|---|---|---|
| Condition | Compound | Name | cParp1/Tubulin | to Hypoxia |
| Control | None | NA | 0.3486621 | 0.49960837 |
| Control + | Forskolin | NA | 0.49535259 | 0.70980557 |
| Forskolin | | | | |
| Hypoxia | none | NA | 0.69787081 | 1 |
| ADM | Original | ADM 1-52 | 0.3980363 | 0.57035814 |
| | peptide | | | |
| | control | | | |
| P5 | Truncated | ADM 13-52 | 0.3846544 | 0.55118281 |
| | agonist | | | |
| | control | | | |
| P12 | Hybrid | ADM 13-22 | 0.33171788 | 0.4753285 |
| | sequence 1 | (cyclical) + | | |
| | | ADM 45-52 | | |
| | | (linear) | | |
| P13 | Hybrid | ADM 13-22 | 0.35347253 | 0.50650138 |
| | sequence 2 | (cyclical) + | | |
| | | ADM 40-52 | | |
| | | (linear) | | |
| P14 | Hybrid | ADM 13-22 | 0.43210566 | 0.61917715 |
| | sequence 3 | (cyclical) + | | |
| | | ADM 35-52 | | |
| | | (linear) | | |
| P15 | Hybrid | ADM 13-22 | 0.66409006 | 0.95159455 |
| | sequence 4 | (cyclical) + | | |
| | | ADM 30-52 | | |
| | | (linear) | | |
| P16 | Hybrid | ADM 13-22 | 0.75822735 | 1.08648669 |
| | sequence 5 | (cyclical) + | | |
| | | ADM 23-52 | | |
| | | (linear) | | |

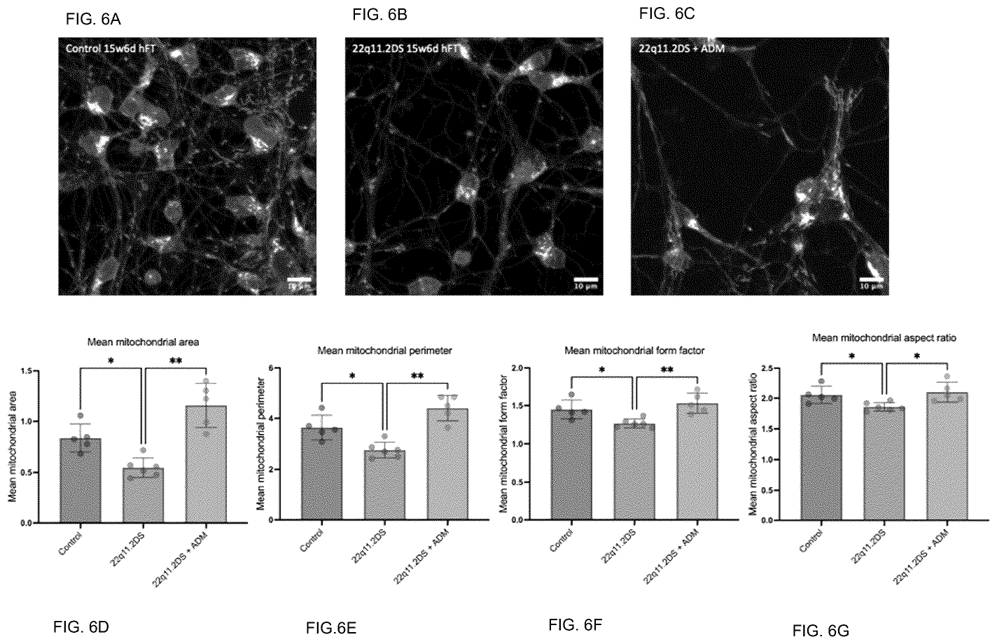

FIG. 6 shows mitochondrial tagging in brain cells from human fetal tissue at 15 weeks 6 days, in control, 22q11.2 deletion and 22q11.2 deletion +ADM conditions; the lower row shows quantified changes in mitochondrial area, perimeter, form factor and aspect ratio in the 3 conditions mentioned above. These results show an improvement in mitochondrial morphology upon exposure of 22q11.2 DS cells to ADM in primary ex vivo human prenatal brain tissue. Peptides were added to the cells for a total of 48 hr, and at a concentration of 0.5 μM.

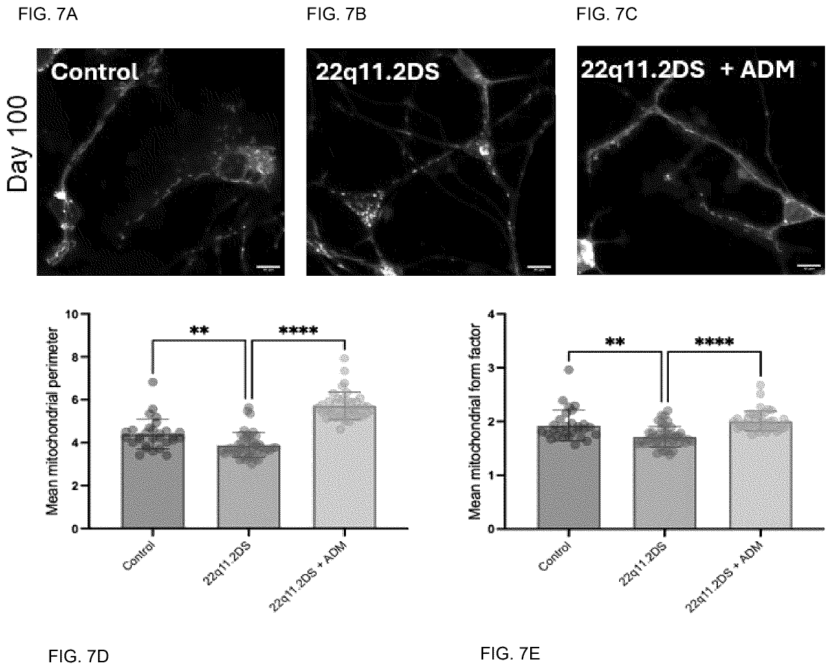

FIG. 7 shows mitochondrial tagging in brain cells from organoids at day 100 in culture, in control, 22q11.2 deletion, and 22q11.2 deletion +ADM conditions; lower row shows quantified changes in mitochondrial perimeter and form factor in the 3 conditions mentioned above. These results show improvement in mitochondrial morphology upon exposure of 22q11.2 DS cells to ADM in organoids.

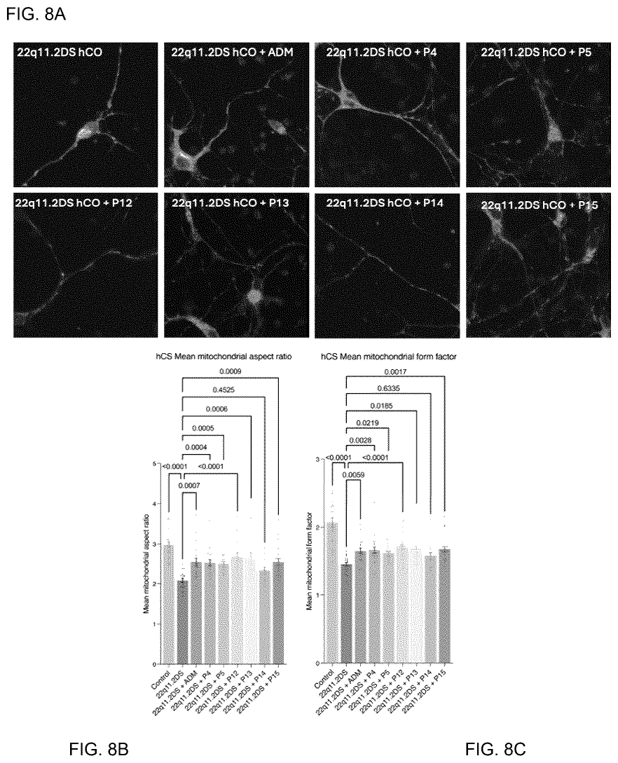

FIG. 8 shows that 22q11.2DS human cortical brain organoids are rescued by ADM analogs. A) Representative micrographs showing mitochondrial morphology visualized using pkmitodeepred dye in monolayer cultures obtained from human cortical brain organoids (hCO) at day 100. Quantification of B) Mean mitochondrial aspect ratio and C) mean mitochondrial form factor. Peptides were added to the cells for a total of 48 hr, and at a concentration of 0.5 μM.

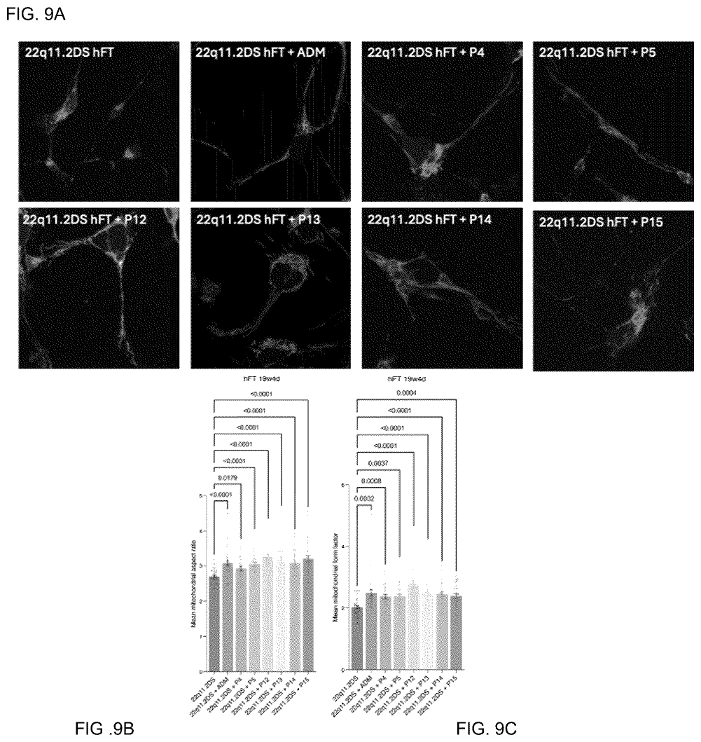

FIGS. 9A-9C. Defects in mitochondria morphology in 22q11.2DS human fetal brain primary neurons are rescued by ADM analogs. A) Representative micrographs showing mitochondrial morphology visualized using pkmitodeepred dye in primary cultures obtained from human fetal brain tissue (hFT) at 19w4d. Quantification of B) Mean mitochondrial aspect ratio and C) mean mitochondrial form factor. Peptides were added to the cells for a total of 48 hr, and at a concentration of 0.5 μM.

### Materials and Methods

Culture of hiPSCs. The hiPSC lines used in this study were validated using standardized methods we previously described. Cultures were regularly tested for, and maintained Mycoplasma free. The differentiation experiments are performed using 4 control hiPSC lines derived from fibroblasts harvested from 4 healthy individuals (two XX and two XY).

Generation of region-specific organoids. Brain region-specific human cortical organoids (hCO) and human subpallium organoids (hSO) were differentiated using feeder-free grown hiPSCs using validated protocols we previously reported and contributed to developing. Briefly, hiPSCs were maintained in six-well plates coated with recombinant human vitronectin (VTN-N, Thermo Fisher Scientific, A14700) in Essential 8 medium (Thermo Fisher Scientific, A1517001). For differentiation, hiPSC colonies were lifted from the plates using Accutase (Innovate Cell Technologies, AT-104). Approximately 3 million cells were transferred to each well in AggreWell 800 (STEMCELL Technologies, 34815) and centrifuged at 100g for 3 minutes in hiPSC medium supplemented with ROCK inhibitor Y-27632 (EMD Chemicals). After 24 hrs, the newly-formed organoids were transferred into ultra-low-attachment plastic dishes (Corning, 430293) in Essential 6 medium (Thermo Fisher Scientific, A1516401) supplemented with dorsomorphin (2.5 μM, Sigma-Aldrich, P5499) and SB-431542 (10 μM, Tocris, 1614) and additional XAV-939 (hCO 0.5 μM, hSO: 2.5 μM Tocris, 3748). This medium was replaced daily for the first five days. On the sixth day in suspension, organoids were transferred to neural medium containing Neurobasal A (Thermo Fisher Scientific, 10888022), B-27 supplement without vitamin A (Thermo Fisher Scientific, 12587010), GlutaMax (2 mM, Thermo Fisher Scientific, 35050061) and penicillin and streptomycin (100 U ml⁻¹, Thermo Fisher Scientific, 15140122). For hCO the neural medium was supplemented with the growth factors EGF (20 ng ml⁻¹, R&D Systems, 236-EG) and FGF2 (20 ng ml⁻¹, R&D Systems, 233-FB) every day until day 15, then every-other day until day 23. For hSO, we included continued supplementation with XAV-939 (2.5 μM) on days 6-23, and SHH pathway agonist SAG (smoothened agonist; 100 nM; Thermo Fisher Scientific, 566660) on days 12-23.

Between days 25-43, the neural medium was supplemented with neurotrophic factors BDNF (20 ng ml⁻¹, Peprotech, 450-02) and NT3 (20 ng ml⁻¹, Peprotech, 450-03), to promote differentiation of the neural progenitors into neurons in both hCO and hSO; media change were performed every other day. After day 43 hCO and hSO were maintained in unsupplemented neural media.

Viral labeling of hSO and generation of human forebrain assembloids (hFA). The viral infection of the hSO for visualization of migrating cortical interneurons was performed as previously described. Briefly, at approximately 45-55 days in culture, hSO were transferred to a 24 well plate (Corning, 3474) containing 500 μl on neural medium with 10 μl of virus (Lenti-DIxi1/2b::eGFP). After 24 hours, 500 μl of neural medium was added. On day 3, all media was removed and replaced with 1 ml of neurobasal medium. The next day, hSO were transferred into fresh neural media in ultra-low attachment plates. After 5-7 days, hCO and virally-infected hSO were used to generate hFA. To do this, one hCO and one hSO were transferred together into a 1.5 ml microcentrifuge Eppendorf tube and maintained in direct contact for three days. More than 95% of hCO and hSO fused. Subsequently, hFA were carefully transferred into 24 well ultra-low attachment plates (Corning, 3474) and media changes were performed very gently every two to three days. Lentivirus construct (lenti-DIxi1/2b::eGFP) was received as a gift from J. L. Rubenstein, and virus was generated by transfecting HEK293T cells with PEI Max (Polysciences, 24765); after 48 hrs the media was collected and ultracentrifuged at 17,000 rpm at 6° C. for 1 hr.

Primary human prenatal cortex processing and viral labeling. Ex vivo human prenatal cerebral cortex at 20 PCW was obtained under a protocol approved by the Research Compliance Office at Stanford University. The tissue was processed within 4 hours after collection using a previously described protocol. In brief, cortical tissue was embedded in 4% low melting-point agarose in PBS and cut using a Leica VT1200 Vibratome at 400 μm. The sections were transferred onto cell culture membrane inserts (diameter, 23.1 mm; pore size, 0.4 μm; Falcon, 353090) and incubated in culture media (66% BME, 25% Hanks, 5% FBS, 1% N-2, 1% penicillin, streptomycin and glutamine; all from Invitrogen) and 0.66% D-(+)-Glucose (Sigma) at 5% CO₂, 37° C. with the DIxi1/2b::eGFP lentivirus for 24 hrs. Sections were then transferred to fresh cell culture media and half media changes were performed every other day. After ˜5 days in culture, DIxi1/2b::eGFP+ cells could be detected and imaging was performed within 7-10 days in culture.

Real time quantitative PCR (qPCR). mRNA was isolated using the RNeasy Mini Kit and RNase-Free DNase set (Qiagen, 74136), and template cDNA was prepared by reverse transcription using the PowerUp SYBR Green master mix for qRT-PCR (Life Technologies, A25742). Real time qPCR was performed on a CFX384 Real time system (BioRad). Data was processed using the BioRad CFX Maestro (BioRad).

Exposure of hSO to hypoxia. At 46 days in culture, 3 hSO from 4 individual hiPSC lines were exposed to hypoxic conditions for 12 and 24 hrs using a C-chamber hypoxia sub-chamber (Biospherix) and Proox 110 Compact Oxygen Controller (Biospherix) with a mixed CO₂/N₂ compressed gas source. For acute induction of hypoxia, the media was previously equilibrated overnight at <1% O₂, 5% CO₂, 37° C. with a resulting PO₂ of 25-30 mmHg. After 12 hrs and 24 hrs of hypoxic exposure, hSO were immediately collected and snap-frozen in dry ice for analyses. Control hSO maintained in regular incubator conditions were collected from each hiPSC line at each experimental timepoint. Additionally, extra hSO exposed to 24 hrs of hypoxia were transferred back to baseline conditions and reoxygenated for 72 hrs. At this timepoint, samples were collected, matched with control hSO and snap-frozen on dry ice.

Synthetic ADM peptide denaturation and LC-MS analysis. The synthetic peptide Adrenomedullin (ADM) (2 nmol, Anaspec, AS-60447) was incubated with either ddH₂0 (for control condition) or Dithiothreitol (DTT) (5 mM, Sigma Aldrich, D5545), at 56° C. for 30 min. Following reduction of disulfide bridges, alkylation of the free SH-groups was performed lodoacetamide (IAM) (20 mM, Sigma-Aldrich, 11149) at 23° C. for 45 min in the dark. The unreacted IAM was quenched with 5 mM final DTT. The solution was desalted and concentrated using Amicon Ultra (3 kD, EMD Millipore, UFC500324), and the volume was adjusted to the starting volume with UltraPure DNase/RNase Free Distilled Water (Thermo Fisher Scientific, 10-977-015). LC-MS analysis was performed using Vanquish LC system coupled with an ID-X Tribrid mass spectrometry equipped with a heated electrospray ionization source (HESI-II, Thermo Scientific). For the LC system, a ZORBAX RRHD diphenyl 100×2.1 mm column (Agilent, 858750-944) was used. The mobile phases were 0.1% formic acid (Fisher Scientific, A117-50) in ddH₂O for A and 0.1% formic acid in acetonitrile (Sigma-Aldrich, 34998) for B. The chromatographic gradient was as follows: 0-2 min 10% B, linear increase 2-10 min up to 60% B, linear increase 10-15 min up to 98% B, hold 15-17 min at 98% B, linear decrease 17-18 min to 10% B, equilibrate 18-21 min at 10% B. The mobile phase flowrate was set to 250 μl/min with an injection volume of 3 μL. The autosampler temperature was set to 4° C., and the column chamber temperature to ambient. Data acquisition on the IDX MS was acquired at 120,000 resolution setting with positive ion voltage of 3.5 kV, vaporizer and transfer tube temperature of 325° C.; AGC target set to standard, RF lens set to 45%, sheath gas set to 40 AU, aux gas set to 12 AU, maximum injection time of 100 ms, with 5 microscans/acquisition, and scan range of 500-2000 m/z. Examination of the LC-MS data was performed using Xcalibur FreeStyle 1.6 (Thermo Scientific).

ADM Analogs. ADM analogs (P12-P16) were synthesized by Biosynth, according to our design instructions for the chemical structure.

Human fetal brain tissue primary culturing, live mitochondria staining and microscopy. Human fetal brain tissues carrying the 22q11.2 deletion at 15 weeks 6 days post conception week and age matched control tissue was acquired from Stanford Hospital. The cerebral cortices from the two tissues were dissected and a single cell suspension was made using a papain-based dissociation method. The cells obtained were plated on Poly-L-Ornithine and Laminin coated cover glass bottom dishes. These primary cultures were maintained in human fetal tissue medium described previously (Michno et al., 2023, biorxiv). After 48 hours of treatment with ADM (0.5 μM), the mitochondria were labeled using the pkmitodeepred dye (Cytoskeleton Inc.) (1:1000 dilution, 15 minutes), washed using fresh medium and imaged on Zeiss LSM980 confocal microscope at 40× magnification.

Mitochondria morphology analyses. Mitochondrial shape was analyzed using the Mitochondria Analyzer plugin in Fiji and the mitochondrial elongation as measure of mitochondrial health is represented as the mitochondrial perimeter, mitochondrial area, form factor and aspect ratio.

ADM testing in 22q11.2 DS organoids and human fetal tissue. Commercially available ADM was added to organoids and human fetal tissue with 22q11.2 DS for a total of 48 hr, and at a concentration of 0.5 μM. Mitochondria parameters were tested using the protocol described above.

ADM and new compounds testing in hypoxia. Commercially available ADM, forskolin (positive control) and the new compounds P12-P16 were added to the hypoxic organoids for a total of 48 hr, according to our published protocols for induction of hypoxia, at a concentration of 0.5 μM.

The ToxiLight™ BioAssay Kit is a bioluminescent, non-destructive cytolysis assay kit designed to measure the release of the enzyme, adenylate kinase (AK), from damaged cells. AK is a robust protein present in all eukaryotic cells, which is released into the culture medium when cells die. The enzyme actively phosphorylates ADP to form ATP and the resultant ATP is then measured using the bioluminescent firefly luciferase reaction. As the level of cytolysis increases, the amount of AK in the supernatant also increases, which results in emission of higher light intensity by the ToxiLight™ reagent. Because the ToxiLight™ BioAssay Kit exploits the fact that AK is released from cells when they die, there is no need for cell lysis (unlike many other cytotoxicity assays). Repeated samples of supernatant can therefore be taken over time without disrupting the cells themselves. This allows for kinetic analysis of cell death.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method of preventing or treating damage to neurons in a subject, the method comprising:
administering an effective dose of adrenomedullin (ADM) or an analog thereof to the subject.

2. The method of claim 1, wherein the method prevents damage to the neurons.

3. The method of claim 1, wherein the damage to neurons results from hypoxia of the brain.

4. The method of claim 1, wherein the damage results from radiation to the brain.

5. The method of claim 1, wherein the damage results from encephalopathy of prematurity.

6. The method of claim 1, wherein the damage results from mitochondrial dysfunction.

7. The method of claim 1 wherein the damage results from a 22q11.2 deletion syndrome.

8. The method of claim 1, wherein the subject is a human neonate or infant.

9. The method of claim 1, wherein the subject is a human child.

10. The method of claim 1, wherein the subject is a human adult.

11. The method of claim 1, wherein the adrenomedullin is while-type human ADM.

12. The method of claim 1, wherein the ADM is an analog stabilized by replacing a disulfide bond with a xylene bridge.

13. The method of claim 12, wherein the analog is truncated relative to wild-type ADM, and which retains at least residues 13-22 and 45-52 of SEQ ID NO:1.

14. The method of claim 12, wherein the analog is truncated relative to wild-type ADM, and which retains at least residues 13-22 and residues 147, P49, G51 and Y52 of SEQ ID NO:1.

15. The method of claim 12, wherein the analog is selected from SEQ ID NO:2, 3, 4, 5, and 6.

16. An adrenomedullin (ADM) analog stabilized by replacing a disulfide bond with a xylene bridge.

17. The analog of claim 16, wherein the analog is truncated relative to wild-type ADM, and which retains at least residues 13-22 and 45-52 of SEQ ID NO:1.

18. The analog of claim 16, wherein the analog is truncated relative to wild-type ADM, and which retains at least residues 13-22 and residues 147, P49, G51 and Y52 of SEQ ID NO:1.

19. The analog of claim 16, selected from SEQ ID NO:2, 3, 4, 5, and 6.

20. A pharmaceutical composition comprising an analog of claim 19, and a pharmaceutically acceptable excipient.
